(19) United States
(12) Patent Application Publication
(10) Pub. No.: US 2019/0110869 A1
(43) Pub. Date: Apr. 18, 2019

(54) PHASE-CHANGING NON-SOLID DENTAL BONE GRAFTING SYSTEM AND METHOD OF USING THE SAME

(71) Applicant: Arpad Alexander Sooky, Aliquippa, PA (US)

(72) Inventor: Arpad Alexander Sooky, Aliquippa, PA (US)

(21) Appl. No.: 16/164,261

(22) Filed: Oct. 18, 2018

Related U.S. Application Data
(60) Provisional application No. 62/573,993, filed on Oct. 18, 2017.

(51) Int. Cl. A61C 8/02; A61F 2/28; A61L 27/12

(52) U.S. Cl. CPC A61C 8/0006; A61F 2/28; A61L 27/12; A61F 2/46; A61L 2430/12; A61F 2310/00293; A61F 2002/2835

(57) ABSTRACT

A bone grafting system is provided. The bone graft material changes phase to a fluid when heated, and so is fluid, gelatinous or otherwise a non-solid physically distinctive form when applied, and that solidifies as it's cooled. Any suitable applicator, such as an eyedropper, can be used to apply the non-solid graft material.

### CROSS-REFERENCE TO RELATED APPLICATION

This application claims the benefit of priority of U.S. provisional application number 62/573,993 filed 18-Oct.-2017, the contents of which are herein incorporated by reference.

### BACKGROUND OF THE INVENTION

The present invention relates to dental bone grafts and, more particularly, to a bone graft system that embodies the application of a liquid, gelatinous, or otherwise non-solid phase graft material, wherein the graft material is adapted to solidify after application.

Bone grafting is a surgical procedure that replaces missing bone and/or promotes bone regeneration. The most common use of bone grafting is in the application of dental implants. Dental implants require bones underneath them for support and proper integration. Some patients, however, have lost portions of their jawbone as a result of dental infection, disease, or trauma.

Current dental bone grafts are typically comprised of small particles in a fluid matrix, which can become dislodged or moved easily during the placement or healing process, thus ruining the intended results. These grafts are also very difficult to pack into tight areas because they are made of particles that may not fit. Current grafts also require a membrane to prevent the fast-moving gingival building cells from arriving to the graft before the slow-moving regenerative bone-building cells, and this barrier membrane can get moved or dislodged easily, allowing the gingival-building cells to form around it. Furthermore, if the fast-moving gingival building cells arrive to the graft before the slow-moving bone-building cells, the graft will turn into gingiva rather than the intended bone.

As can be seen, there is a need for an improved dental bone graft that delays gingival-building cells migrating into it, and gives bone-building cells a better opportunity to arrive there and turn it into bone, while also making handling easier, improving application into tight spaces, and preventing dislodgement or deformation after/during placement. The bone grafting system embodied in the present invention may include a solid dental bone graft that liquifies when heated, and solidifies when cooled to body temperature. Another application is a multiple component bone graft that is liquid immediately after being mixed, and the mixing produces a reaction that solidifies the bone graft in a certain amount of time. The bone graft can contain any number or combination of growth factors.

### SUMMARY OF THE INVENTION

In one aspect of the present invention, graft material for grafting bone to human tissue, includes a graft material having a non-solid physically distinctive form at a time of application, wherein the grafting material solidifies thereafter in a procedurally suitable amount of time.

In another aspect of the present invention, graft material for grafting bone to human tissue includes a graft material having a non-solid physically distinctive form at a time of application, wherein the grafting material solidifies thereafter in a procedurally suitable amount of time, wherein the melting temperature of the graft material is higher than body temperature, and wherein the phase state of the graft material is fluid below 47 degrees Celsius, wherein the graft material does not comprise particulate at the time of application, and wherein the graft material comprises one or more growth factors selected from a group consisting of collagen, hydroxyapatite, allograft, and xenograft.

In yet another aspect of the present invention, a method of grafting bone to human tissue includes applying the above-mentioned graft material to a portion of human tissue to be treated; and cooling through a cooling device or otherwise allow said graft material to cool into a solid state.

These and other features, aspects and advantages of the present invention will become better understood with reference to the following drawings, description and claims.

### BRIEF DESCRIPTION OF THE DRAWINGS

FIG. 1 is a schematic view of an exemplary embodiment of the present invention, illustrating the implant prior to treatment;

FIG. 2 is a schematic view of an exemplary embodiment of the present invention, illustrating the implant prior to treatment;

FIG. 3 is a flowchart of an exemplary embodiment of the present invention; and

FIG. 4 is a schematic view of a grafting material of exemplary embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, one embodiment of the present invention is a solid dental graft that changes phase to a fluid when heated, and so is fluid, gelatinous or otherwise non-solid physically distinctive form when applied, and that solidifies as it's cooled. Any suitable applicator, such as an eyedropper, can be used to apply the non-solid graft material.

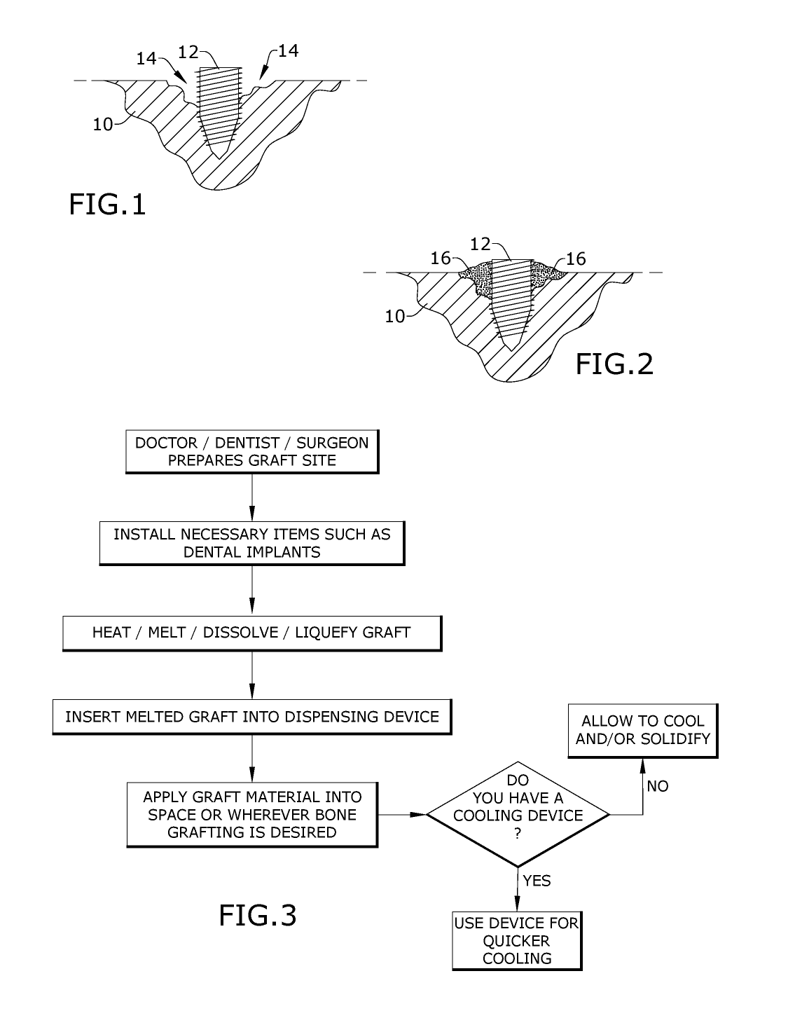

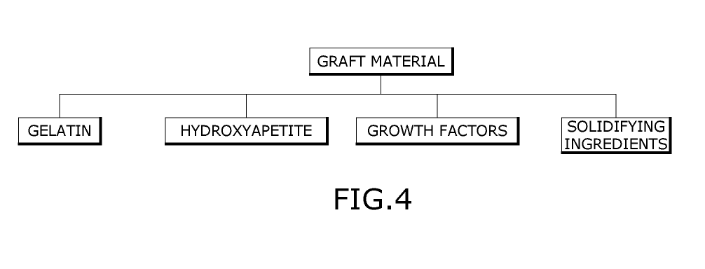

Referring to FIGS. 1 through 4, the present invention may include a bone graft system that embodies a graft material 16 in a non-solid physically distinctive form during application, such as a liquid or gelatinous form, wherein the graft material 16 is adapted to solidify into a solid phase post application.

The melting temperature of the graft material 16 is higher than body temperature, so that after the fluid graft is placed during, it gelatinizes and turns solid when cooled to body temperature, thereby making its dislodgement or deformation less possible. Advantageously, it also prevents gingiva building cells from migrating into the graft and converting the graft into gingival tissue rather than osseous tissue, because the solid makes it more difficult for gingival cell migration.

Since it is a fluid when applied, the graft material 16 is easily squirted or placed into tight areas that are difficult to pack particulate bone graft into. No other bone grafts are in existence that liquify when heated and solidify after cooling to body temperature.

Unlike current bone graft material, that are very difficult to keep all the tiny particles of existing bone graft material in place while placing multiple membranes and suturing/closing the surgical site. The claimed graft locks into the intended place when it solidifies, sealing the area from migrating gingival-building cells, thereby making not only the surgery easier, but the patient less likely to accidentally dislodge/disform the graft during the healing process, or the graft converting to gingival tissue rather than bone.

Furthermore, because the graft material solidifies after placement, gingival-building cells take longer to migrate into/through it, giving bone-building cells a better opportunity to arrive there and turn it into bone instead. It also stays in place and keeps its shape once solidified, thereby making surgery, wound closure, and the healing process easier and more predictable.

In one embodiment, the graft material 16 itself can be composed of many different substances such as collagen, hydroxyapatite, allograft, xenograft, or the like, so long as it turns fluid below 47 degrees Celsius, yet above human body temperature.

In one embodiment, a heating device is used, which keeps the graft at exactly the right temperature for it to be fluid, yet not so hot that it would cause thermal damage to tissue when placed.

In another embodiment, the heating device contains a tray where the solid bone graft is placed. After it liquefies, the applicator is used to carry the graft to the intended surgically prepared graft site. After placement, the optional cooling device can be used to cool and solidify the graft. The surgical site is then closed.

In an alternative embodiment, an optional device which cools and solidifies the graft on demand can be used, rather than having to wait for it to cool to body temperature on its own.

In another embodiment, the bone graft does not need to be heated at all, but its constituents are fluid before being mixed together. The mixing of the components produces a reaction which solidifies the mixed bone graft in a certain amount of time, giving time for the mixed components to be applied to the intended grafting site while still in the liquid phase and before it has solidified.

In operation, a dental surgical site 14 is prepared, which may have an osseous defect and is intended to be grafted (augmented) with bone. The heating device liquefies the bone graft material 16, which is then placed into or onto the associated tissue 10 where bone is needed. If the surgeon does not want to wait for the graft to cool and solidify on its own, the optional cooling device can be applied to the graft, which speeds up the solidification of the graft. Alternatively, the components of the graft are mixed and then placed into or onto the operative tissue 10 where bone is needed while still in liquid form, where it then solidifies after a suitable amount of time has passed. The non-solid graft material 16 is particularly suited for into tight spaces such as around dental implants 12, placed immediately into the socket after pulling a tooth out. The surgical site 14 is then closed with sutures, additional membranes or barriers, or however the surgeon deems fit.

In order to create the graft material 16 for distribution, the graft material 16 ingredients need to be liquefied and mixed thoroughly, then poured into an appropriate container for sale. The graft material 16 itself can consist of any number of ingredients such as collagen, which makes a suitable matrix for bone growth, or include any growth hormones which stimulate bone formation. In an alternative embodiment, the graft material ingredients need to simply be mixed together, then poured or applied to the grafting site. In another alternative embodiment, the graft can be used on any other part of the body where bone is needed, or on non-human subjects that need bone grafts.

It should be understood, of course, that the foregoing relates to exemplary embodiments of the invention and that modifications may be made without departing from the spirit and scope of the invention as set forth in the following claims.

What is claimed is:

1. A graft material for grafting bone to human tissue, comprising:
a graft material having a non-solid physically distinctive form at a time of application, wherein the grafting material solidifies thereafter in a procedurally suitable amount of time.

2. The graft material of claim 1, wherein the non-solid physically distinctive form is gelatinous.

3. The graft material of claim 1, wherein the melting temperature of the graft material is higher than body temperature.

4. The graft material of claim 1, wherein the phase state of the graft material is fluid below 47 degrees Celsius.

5. The graft material of claim 1, wherein the graft material does not comprise particulate at the time of application.

6. The graft material of claim 1, wherein the graft material comprises one or more growth factors selected from a group consisting of collagen, hydroxyapatite, allograft, and xenograft.

7. A graft material for grafting bone to human tissue, comprising:
a graft material having a non-solid physically distinctive form at a time of application, wherein the grafting material solidifies thereafter in a procedurally suitable amount of time, wherein the melting temperature of the graft material is higher than body temperature, and wherein the phase state of the graft material is fluid below 47 degrees Celsius,
wherein the graft material does not comprise particulate at the time of application, and
wherein the graft material comprises one or more growth factors selected from a group consisting of collagen, hydroxyapatite, allograft, and xenograft.

8. A method of bone grafting, comprising the steps of:
applying a graft material of claim 1 to a portion of human tissue to be treated; and
allowing said graft material to cool into a solid state.

9. The method of claim 8, further comprising the step of:
providing a cooling device to cool the graft material.
